(19) United States
(12) Patent Application Publication
(10) Pub. No.: US 2026/0123999 A1
(43) Pub. Date: May 7, 2026

(54) ELECTRONICALLY GUIDED PRECISION MEDICAL TARGETING USING NEAR INFRARED FLUORESCENCE IMAGING

(71) Applicant: Johnson & Johnson Enterprise Innovation Inc., New Brunswick, NJ (US)

(72) Inventors: Alexandru Paunescu, Clinton, NJ (US); Matthew Krever, Basking Ridge, NJ (US); Michelle Mahoney, MIddletown, RI (US); Chad E. Eckert, Terrace Park, OH (US); Christopher Sramek, Half Moon Bay, CA (US); Charles Scheib, Loveland, OH (US); Ravi Patel, Rochester, NY (US)

(21) Appl. No.: 19/117,843

(22) PCT Filed: Nov. 10, 2023
(86) PCT No.: PCT/IB2023/061385

§ 371 Date: Apr. 2, 2025

Related U.S. Application Data
(60) Provisional application No. 63/383,235, filed on Nov. 10, 2022.

(51) Int. Cl. A61B 34/20; A61B 1/00; A61B 1/04; A61B 34/10; A61B 34/37; A61B 90/00

(52) U.S. Cl. CPC A61B 34/20; A61B 1/000094; A61B 1/043; A61B 34/10; A61B 34/37; A61B 90/361; A61B 2034/107; A61B 2034/2051

(57) ABSTRACT

A medical targeting system facilitates guidance of a real-time imaging system and intervention tool to a calibrated position of a target identified in preprocedural images. Real-time images from an imaging system are registered with preprocedural images. The imaging device is guided to the vicinity of a virtual target identified from the registered preprocedural images until the physical target is identified in the real-time images. The virtual target position can then be calibrated to correspond to the physical target. Once calibrated, an intervention tool can be guided to the calibrated position to perform the procedure, even if the real time imaging no longer shows physical target. The imaging device may include a near infrared fluorescence (NIRF) fiberscope that enables imaging of targets outside of the anatomical channel or other pathway being traversed by the imaging device.

### BACKGROUND

### Technical Field

The described embodiments relate to a system and method for precision guidance of medical tools to an anatomical target.

### Description of Related Art

Successful robotic-guided diagnosis and therapy depends on the ability to obtain precise anatomical positions of a tumor or other anatomical target. Typically, such systems rely on identification of the target position in preprocedural images (e.g., computerized tomography (CT) scan images) obtained from a preprocedural imaging system. The preprocedural images are then registered to the real-time anatomy of the patient during the procedure based at least in part on anatomical landmarks, electromagnetic sensors, or other techniques. However, this registration process may suffer from inaccuracies due to anatomical changes in the patient between the preprocedural image scan and the procedure, noise in the electromagnetic field affecting the electromagnetic sensors, or other factors. Such inaccuracies complicate the procedure and may negatively impact the likelihood of success.

### SUMMARY

A medical targeting system performs calibrated guidance to a physical target.

Preprocedural images representing an anatomy are obtained. An initial virtual target position of the physical target in the preprocedural images is determined. Real-time intraprocedural images of the anatomy are furthermore obtained from a real-time imaging device. The real-time intraprocedural images of the anatomy are registered to the preprocedural images to generate a mapping between coordinates of the real-time intraprocedural images and coordinates of the preprocedural images. The real-time imaging device is electronically guided to a vicinity of the initial virtual target position using the mapping. A position of the physical target is obtained and a calibrated virtual target position corresponding to the obtained position of the physical target is determined. Guidance of a medical tool to the physical target is facilitated based at least in part on the calibrated virtual target position.

In an embodiment, determining the calibrated virtual target position comprises presenting, to a display device, a representation of a virtual target at the initial virtual target position overlaid on the real-time intraprocedural images, receiving at least one user input for repositioning the virtual target and updating the display device to indicate the repositioning, and determining the position of the physical target responsive to detecting alignment between a repositioned virtual target and the physical target.

In an embodiment, the at least one user input comprises at least one of a first input comprising a first adjustment of the virtual target along a first axis, a second input comprising a second adjustment of the virtual target along a second axis, and a third input comprising a third adjustment of the virtual target along a third axis.

In an embodiment, determining the calibrated virtual target position comprises generating a representation of a virtual target at the initial virtual target position overlaid on the real-time intraprocedural images, automatically generating adjustments to the initial virtual target position and applying an image analysis until the virtual target substantially overlaps the physical target, and determining the position of the physical target based at least in part on the adjustments.

In an embodiment, determining the calibrated virtual target position comprises receiving user inputs to control position of the real-time imaging device to capture at least two images of the physical target from different viewpoints, and determining the position of the physical target based at least in part on the at least two images.

In an embodiment, determining the position of the physical target comprises receiving user inputs marking the physical target in the at least two images.

In an embodiment, determining the position of the physical target comprises automatically identifying the physical target in the at least two images using image analysis.

In an embodiment, facilitating the guidance comprises generating electronic navigation guidance for an electronically assisted intervention tool to the calibrated virtual target position.

In an embodiment, facilitating the guidance comprises displaying, on a display screen, the preprocedural images and a representation of a virtual target at the calibrated virtual target position overlaid on the preprocedural images and displaying, on the display screen, a position of an intervention tool overlaid on the preprocedural images to enable guidance of the intervention tool to the calibrated virtual target position.

In an embodiment, facilitating the guidance comprises displaying, on a display screen, the real-time intraprocedural images and a representation of a virtual target at the calibrated virtual target position overlaid on the real-time intraprocedural images.

In an embodiment, facilitating the guidance comprises determining a distance from an intervention tool to the physical target, and generating control signals to guide the intervention tool based at least in part on the distance.

In an embodiment, the real-time imaging device comprises an endoscope for obtaining visible light images.

In an embodiment, the real-time imaging device further includes a fiberscope for obtaining near-infrared fluorescent images. Registering the real-time intraprocedural images to the preprocedural images may comprise registering the visible light images from the endoscope to the preprocedural images and registering the near-infrared fluorescent images from the fiberscope to the visible light images from the endoscope.

In an embodiment, the endoscope is a bronchoscope.

In an embodiment, the fiberscope has a substantially fixed relative position to the endoscope, Registering the near-infrared fluorescent images to the visible light images may be based at least in part on the substantially fixed relative position.

In an embodiment, the fiberscope is positioned within a field of view of the endoscope. Registering the near-infrared fluorescent images to the visible light images may be based at least in part on detecting the fiberscope in the visible light images.

In an embodiment, the endoscope is positioned within a field of view of the fiberscope. Registering the near-infrared fluorescent images to the visible light images may be based at least in part on detecting the endoscope in the near-infrared fluorescent images.

In an embodiment, the fiberscope is configured to pass through a working channel of the endoscope.

In an embodiment, obtaining the real-time intraprocedural images of the anatomy further comprises obtaining a time-of-flight of near-infrared fluorescent light projected by a fiberscope to determine a depth component of near-infrared fluorescent images.

In an embodiment, determining the calibrated virtual target position corresponding to the detected position of the physical target comprises emitting, by a fiberscope of the real-time imaging device, near-infrared light of one or more wavelength, detecting, by the fiberscope, reflectance properties of tissue in a path of the near-infrared light, and discerning when the physical target is in the path of the near-infrared light based at least in part on the reflectance properties.

In an embodiment, determining the detected position of the physical target is based at least in part on a time-of-flight of the near-infrared light between the fiberscope and a surface of the physical target.

In an embodiment, the method further comprises determining a distance from the real-time imaging device and a surface of obscuring tissue based at least in part on a time-of-flight of white light emitted from a white light illuminator of the real-time imaging device and reflected by the surface of the obscuring tissue, and determining a distance from the surface of obscuring tissue and the surface of physical target.

In another embodiment, a method is provided for performing a medical procedure in a patient. An initial virtual target position of a physical target is identified from preprocedural images of the patient. An imaging system is inserted into a patient to capture real-time intraprocedural images of the patient. The imaging system is navigated to a vicinity of the initial virtual target position of the physical target using electronically generated guidance based in part on a registration between the real-time intraprocedural images and the preprocedural images.

Responsive to detecting the physical target in the real-time intraprocedural images, a calibration is performed to identify a calibrated virtual target position corresponding to a detected position of the physical target in the real-time intraprocedural images. Based at least in part on the calibrated virtual target position, the medical procedure associated with the physical target is performed using an intervention tool.

In an embodiment, the imaging system comprises a bronchoscope for capturing visible light images as a component of the real-time intraprocedural images. The bronchoscope may include a working channel. The imaging system may further comprise a fiberscope insertable in the working channel of the bronchoscope. The fiberscope may capture near infrared fluorescence images as a component of the real-time intraprocedural images.

In an embodiment, performing the medical procedure comprises withdrawing the fiberscope from the working channel of the bronchoscope, and introducing the intervention tool into the working channel of the bronchoscope.

In further embodiment, a non-transitory computer-readable storage medium stores instructions for performing any of the above-described methods. The instructions when executed by one or more processors cause the one or more processors to perform steps for facilitating the methods described herein.

In a further embodiment, a medical targeting system comprises a real-time imaging device, a medical tool, one or more processors, and a non-transitory computer-readable storage medium storing instructions for performing guidance to a physical target. The instructions when executed by the one or more processors cause the one or more processors to perform steps for facilitating any of the methods described herein.

In an embodiment, the medical tool comprises an electronically navigated intervention tool, and the medical targeting system further comprises a robotic control system for controlling electronic navigation of the electronically navigated intervention tool.

### BRIEF DESCRIPTION OF THE DRAWINGS

FIG. 1 is an example embodiment of a medical environment for an electronically-assisted medical procedure.

FIG. 2 is an external view of an airway showing a virtual target estimated from preprocedural images relative to position of a real target, prior to calibration.

FIG. 3 is an example embodiment of a first calibration technique for calibrating position of a virtual target in association with performing an electronically assisted medical procedure.

FIG. 4 is an example embodiment of a second calibration technique for calibrating position of a virtual target in association with performing an electronically assisted medical procedure.

FIG. 5 is an example embodiment of a technique for discerning and estimating distances between a tumor and obscuring tissue.

FIG. 6 is an example embodiment of a process for performing an electronically assisted medical procedure using a calibration technique for calibrating position of a virtual target representing a tumor or other physical target.

FIG. 7 is an example embodiment of a process for calibrating a position of a virtual target representing a tumor or other physical target in association with an electronically assisted medical procedure.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

An electronically assisted medical procedure is performed by facilitating guidance of a real-time imaging system and intervention tool to a calibrated position of a target identified in preprocedural images. After positioning the patient for the medical procedure, real-time images from an imaging system are registered with preprocedural images. The imaging device is guided to a vicinity of a virtual target identified from the registered preprocedural images until the physical target is identified in the real-time images. The virtual target position can then be calibrated to correspond to the detected position of the physical target. Once calibrated, an intervention tool can be guided to the calibrated position to perform the procedure. The imaging device may include a near infrared fluorescence (NIRF) fiberscope that enables imaging of targets outside of the airways or other anatomical features being traversed by the imaging device. The imaging device may furthermore enable discernment between a tumor and obscuring tissue, and estimation of relative distances between surfaces of the tumor and obscuring tissue.

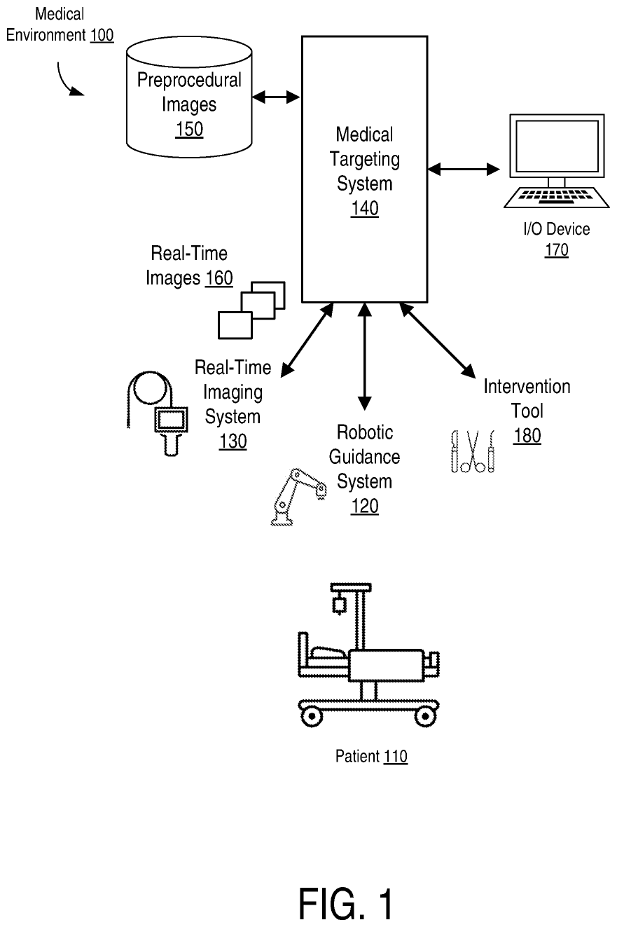

FIG. 1 illustrates an example embodiment of a medical environment 100 for performing a medical procedure on a patient 110. The medical environment 100 includes a medical targeting system 140, a robotic guidance system 120, a real-time imaging system 130 for capturing real-time images 160, one or more intervention tools 180, a preprocedural image datastore storing preprocedural images 150, and one or more input/output (I/O) devices 170. In different variations, the medical environment 100 may include different or additional components.

The medical targeting system 140 and preprocedural image datastore storing the preprocedural images 150 may be all or partially co-located with the patient 110 (e.g., in an operating or examination room of a medical facility), or may be at least partially located remotely (e.g., in a server room of the medical facility or in a cloud server system remote from the medical facility). The various electronic components 120, 130, 140, 150, 170 may all or partly be communicatively coupled via one or more networks (e.g., a local area network (LAN), wide area network (WAN), cellular data network, or combination thereof), and/or may be directly communicatively coupled via wired or wireless communication links (e.g., WiFi direct, Bluetooth, Universal Serial Bus (USB), or other communication link).

The preprocedural images 150 comprise a set of images of the patient 110 captured prior to the medical procedure. The preprocedural images 150 may comprise, for example, CT scan images, magnetic resonance imaging (MRI) images, ultrasound images, X-ray images, positron emission tomography (PET) images, or other images or combinations thereof relevant to the procedure. In an embodiment, the preprocedural images 150 may comprise volumetric images that provide a visual representation of an anatomy in three-dimensional space. Such volumetric images may be captured and stored as a set of parallel image slices each representing a two-dimensional plane. The volumetric images may be rendered (e.g., via a display of the I/O device 170) as discrete two-dimensional image slices or via a perspective view that is perceived by the viewer as a three-dimensional structure. The preprocedural images 150 may also include a virtual three-dimensional model of the imaged anatomy that may be generated based at least in part on the raw images.

The preprocedural images 150 may be annotated to indicate one or more target positions associated with an anatomical target (e.g., a tumor or other nodule, lesion, or other anatomical structure) of the procedure. The position may be specifically marked in the preprocedural images 150, or may be represented by a set of coordinates (e.g., x, y, z coordinates) in the three-dimensional image space. The marked position may correspond to a centroid of the target. Optionally, the annotations may include other characteristics of the anatomical target such as its size, shape, visual characteristics, or other identifying information. The annotations may be stored as metadata associated with the preprocedural images 150 or may be stored in a separate data structure associated with the preprocedural images 150.

The real-time imaging system 130 captures real-time images 160 of the patient 110 during the medical procedure. The real-time imaging system 130 may comprise an endoscope having one or more light sources and one or more light sensors (e.g., cameras) coupled to a probe tip of a long, thin, tube (e.g., rigid or flexible) that can be threaded through various anatomical channels such as airways, blood vessels, gastrointestinal tract, or other channels, or other pathways (such as through tissue) including those that may be formed by a needle, cannula, or other instrument. The real-time imaging system 130 may furthermore include one or more sensors (e.g., an electromagnetic coil) proximate to the probe tip that enables a position-sensing system to detect a position of the probe tip as it traverses through the anatomy.

The probe tip of the real-time imaging system 130 may comprise a visible light camera (e.g., single camera, stereoscopic camera, or multi-view camera) that captures visible light images, and/or a near infrared fluorescence (NIRF) fiberscope. The NIRF fiberscope may include an illuminator that projects near infrared light to excite fluorescence to either an endogenous or exogeneous fluorophore, which is sensed by a NIRF sensor. Depending on the wavelength, the NIRF light may penetrate certain types of tissue or may be reflected by other types of tissue with varying levels of reflectance. By tuning the NIRF illuminator based at least in part on these varying reflectance characteristics, the NIRF probe can enable detection of structures both on a surface of an anatomical feature (e.g., an anatomical channel) or behind the surface. For example, in a bronchoscopy, the NIRF images may depict tumors that are on the other side of the airway wall from where the NIRF fiberscope is located. In some embodiments, a fluorescent dye may be introduced to the anatomy to enable or supplement fluorescence detected by the NIRF fiberscope. In other embodiments, the NIRF fiberscope may detect endogenous fluorophores without utilizing any fluorescent dye.

The reflective properties of tissue illuminated by the NIRF illuminator may be detected by the NIRF sensor and analyzed to discern between different types of tissue, such as between a tumor, connective tissue, functional tissue, or other tissues. For example, at certain wavelengths, the reflectance of obscuring tissue may be relatively low (i.e., the NIRF light mostly passes through the tissue) relative to the reflectance of a tumor. The reflectance properties can thus be measured to detect when the beam is directed at a tumor or other target tissue type.

In an embodiment, a multispectral discernment technique may be employed in which the NIRF illuminator is controllable to sequentially emit photons of multiple different wavelengths. The reflected photons may then be detected, and the reflectance properties measured for the different wavelengths and analyzed to discern the tissue type. For example, the reflectance properties at different wavelengths can be compared with known reflectance characteristics of target tissue types at each of the different wavelengths to determine whether a certain a target tissue type (e.g., a tumor) is in the path of the NIRF beam. In one embodiment, a multispectral technique may be used in which a pair of wavelengths are emitted that have known discernable reflective properties associated with one or more tissue types. In other embodiments, three or more different wavelengths may be utilized. In further embodiments, a single wavelength may be used. In yet further embodiments, a Raman spectroscopy technique may be employed for tissue discernment or classification.

In an embodiment, the NIRF fiberscope and/or the white light camera may enable distance estimations between the probe tip and the detected tissue based at least in part on time-of-flight of the light projected by the illuminator and the reflected light sensed by the NIRF sensor and/or white light image sensor. In a further embodiment, the probe tip of the real-time imaging system 130 may include a separate laser-based time-of-flight sensor. The sensor may optionally be used to detect distances between the probe tip and a structure, such as a target tumor or other discernable tissue type. In a further embodiment, the real-time imaging system may employ a Fourier Transform Infrared (FTIR) technique in which an infrared spectrum of absorption or emission of a target structure is measured in order to estimate distance to the target.

In an embodiment, similar techniques may be employed to discern between tissue and non-anatomical structures such as an intervention tool, implanted device, or other element.

For example, the same multispectral discernment technique may distinguish between reflectance properties of tissue (or a specific type of tissue) and other non-anatomical materials that may have been artificially introduced into the anatomy. Similarly, distances to non-anatomical targets may be estimated using any of the distance estimation techniques described herein.

In a particular configuration, the real-time imaging system 130 includes a combination of a visible light camera and a NIRF fiberscope. Here, the visible light camera may be integrated in an endoscope (e.g., a bronchoscope) having a tube with one or more working channels that allows for insertion of a NIRF fiberscope. Alternatively, the visible light camera may comprise a separate instrument movable in a working channel of a tube that is insertable into the anatomy. In some embodiments, the NIRF fiberscope can be removed from the working channel and an intervention tool 180 could be inserted in its place. The intervention tool 180 may comprise any device that facilitates a medical procedure such as a biopsy needle, cutting device, sensing device, medicant delivery device, or other deployable tool. The endoscope could also have multiple working channels that enable both the NIRF fiberscope and one or more intervention tools 180 or other sensing devices to be inserted concurrently. For example, in some embodiments, an additional working channel may be used for a white light illuminator or an additional NIRF illuminator having either the same or different wavelength than the NIRF illuminator of the NIRF fiberscope. In further embodiments, the real-time imaging system 130 may include a visible light camera and a NIRF fiberscope that are integrated into a single probe tip. Furthermore, in some embodiments, multiple instruments could pass through the same working channel of the endoscope.

The visible light camera and NIRF fiberscope may be physically aligned in various ways. For example, in one configuration, the NIRF fiberscope is freely positioned within a working channel of the endoscope such that the NIRF fiberscope can freely move relative to the visible light camera along the longitudinal axis of the working channel. In another configuration, the NIRF fiberscope may be physically constrained to a fixed axial position relative to the visible light camera. For example, the NIRF fiberscope may be constrained to be substantially flush with the visible light camera, extended by a fixed distance past the position of the visible light camera (such that the NIRF fiberscope is within the field of view of the visible light camera), or recessed by a fixed distance relative to the visible light camera (such that the visible light camera and/or the interior walls of the working channel are within the field of view of the NIRF fiberscope).

The robotic guidance system 120 facilitates movement of the real-time imaging system 130 through one or more anatomical channels or other pathways associated with anatomical features of the patient 110. The robotic guidance system 120 may comprise a robotic-assisted device that facilitates control and navigation of the real-time imaging system 130 responsive to control inputs provided by an operator (e.g., a medical professional) using a I/O device 170 (e.g., a handheld controller or other computer input device). Alternatively, the robotic guidance system 120 may provide automated guidance in which it directly navigates the real-time imaging system 130 to the anatomical target without necessarily requiring manual control inputs. Here, the robotic guidance system 120 may facilitate navigation based at least in part on a predefined pathway or the robotic guidance system 120 may automatically determine the pathway.

The robotic guidance system 120 may enable real-time tracking of the probe tip of the real-time imaging system 130 using electromagnetic tracking, image-based tracking (including white light and/or fluorescent light tracking), shape sensing, or other techniques. Thus, the real-time position of the probe tip (which corresponds approximately to the position of the visible light camera and/or NIRF fiberscope of the real-time imaging system 130) can be tracked.

The robotic guidance system 120 may similarly facilitate navigation of one or more intervention tools 180 to a target position. This guidance may be facilitated using electromagnetic sensors attached to or proximate to the intervention tool 180, image-based tracking of the intervention tool 180 (using the real-time images 160), or other tracking mechanisms. The robotic guidance system 120 may furthermore control actuation of the intervention tool 180 (e.g., deployment of a needle). In an embodiment, the intervention tool 180 and/or the real-time imaging system 130 deployed together with the intervention tool 180 may detect a distance from the intervention tool 180 to the target during the procedure (e.g., based at least in part on image analysis or a time-of-flight system) to enable precise deployment of the intervention tool 180 to the target.

In different embodiments, the robotic guidance system 120 may operate with different levels of autonomy. For example, in some embodiments, the robotic guidance system 120 is entirely under control of a human operator that controls movements using the I/O device 170. In other embodiments, the robotic guidance system 120 may operate in conjunction with the medical targeting system 140 to perform completely automated procedures based at least in part on the target marked in the preprocedural images 150. Other embodiments may include a combination of manually controlled and automatically controlled functions.

The medical targeting system 140 may obtain both the preprocedural images 150 and the real-time images 160 from the real-time imaging system 130 and generates guidance data to facilitate navigation of the real-time imaging system 130 during the medical procedure. The guidance data may control the robotic guidance system 120 or may be presented via the I/O device 170 to enable manual guidance by a medical professional.

The medical targeting system 140 may perform a registration between the set of preprocedural images 150 and the real-time images 160 to map between respective coordinates of the image spaces in the preprocedural images 150 and the real-time images 160. In this way, the real-time position of the probe end of the real-time imaging system 130 can be mapped to a specific position in the image space of the preprocedural images 150, or vice versa. Furthermore, the target position of the anatomical target in the preprocedural images can be mapped to a virtual target position in the real-time images. Registration may be aided by various motion filtering techniques that may be based at least in part on external data sources relating to patient motion. For example, ventilator data could provide information about inflation and deflation of the lung that can be filtered out in the registration process. Furthermore, sensor data from various sensors (e.g., accelerators, electromagnetic sensors, pressure transducers, etc.) may detect motion of the patient that can be used to improve registration accuracy.

In an embodiment where the real-time imaging system 130 includes both a visible light camera and a NIRF fiberscope, registration may occur by first registering the visible light images with the preprocedural images 150, and then registering the NIRF images with the visible light images. In this way, a common set of coordinates may be established between all sets of images. Registering the NIRF images to the visible light images may be dependent on the physical alignment between the NIRF fiberscope and the visible light camera. For example, if the NIRF fiberscope and visible light camera are constrained to be flush with each other, the respective images may be registered by compensating for an offset between their respective positions. If the NIRF fiberscope is extended relative to the visible light camera, registration may be performed based in part on performing image analysis to identify the position of the NIRF fiberscope in the field of view of the visible light images. If the NIRF fiberscope is recessed relative to the visible light camera, registration may be performed based in part on performing image analysis to identify the position of the visible light camera in the field of view of the NIRF images.

Positions of intervention tools 180 may similarly be registered to the real-time images 160 based at least in part on predefined physical alignment between the intervention tool 180 and the probe tip of the real-time imaging system 130, electromagnetic tracking, image-based tracking, or a combination thereof. Thus, real-time positions of the intervention tool 180 may be tracked in the image space of the real-time images 160 and may be mapped to positions in the preprocedural images 150 (direct images or a virtual model derived from them) based at least in part on the registration between the preprocedural images 150 and the real-time images 160.

The medical targeting system 140 may furthermore determine a navigation path to the virtual target position representing the position of the target in the preprocedural images 150 as mapped to the real-time images. For example, the medical targeting system 140 may generate control signals for controlling navigation of the real-time imaging system 130 to a vicinity of the virtual target position (either automatically or based at least in part on human inputs). Once the physical target is in view of the real-time imaging system 130, a discrepancy may be observed between the actual observed position of the anatomical target and the virtual target position that estimates its position. This discrepancy may result from anatomical changes in the patient that occurred after capturing the preprocedural images, noise in the electromagnetic field affected the electromagnetic sensors, or other factors. To resolve the discrepancy, the medical targeting system 140 may perform a calibration of the virtual target position to align the virtual target position to the position of the observed target. Example of calibration techniques are described in further detail below.

After calibrating the virtual target position, an intervention tool 180 may be deployed to the calibrated virtual target position to perform the procedure. The intervention tool may comprise, for example, a biopsy needle or other device at an end of a long flexible tube that can traverse through an anatomical channel or other pathway. The intervention tool 180 may be controlled and/or tracked by the robotic guidance system 120 similar to the real-time imaging system 130. Here, the real-time imaging system 130 (or components thereof) may be optionally removed prior to deploying the intervention tool 180. In this case, guidance to the calibrated virtual target position may be facilitated based at least in part on electromagnetic guidance or other electronic guidance without necessarily utilizing concurrent real-time imaging. In another embodiment, the intervention tool 180 may be deployed together with the real-time imaging system 130. For example, the intervention tool 180 may operate through a working channel of an endoscope.

The medical targeting system 140 may be implemented using on-site computing and/or storage systems, cloud computing and/or storage systems, or a combination thereof. Accordingly, the medical targeting system 140 may be local, remote, and/or distributed with portions being local and portions remote, where the various system elements may be communicatively coupled over a network. The medical targeting system 140 may implement the functions described herein by one or more processor and a non-transitory computer-readable storage medium that stores instructions executable by the one or more processors to perform the described functions.

The I/O device 170 may comprise any display and/or electronic input device. The display may comprise a computer display screen, touchscreen for a laptop, tablet, or mobile device, a television screen, projector, virtual or augmented reality head mounted display or goggles, or other display. The input device may include any computer input device such as a keyboard, mouse, joystick, game controller, touchscreen voice recognition system, gesture recognition system, or other input device. The display and input device may be integrated (e.g., as in a tablet or mobile device) or may comprise separate devices.

The I/O device 170 may render various views of the preprocedural images 150 and/or the real-time images 160 to facilitate the medical procedure. Various virtual objects and/or other data may optionally be overlaid on the preprocedural images 150 and/or the real-time images 160. For example, a position of the virtual target marked in the preprocedural images and registered to the real-time images 160 may be rendered as a virtual object in a display of the real-time images 160. Furthermore, a tracked position of the probe tip of the real-time imaging system 130 may be mapped to coordinates in the preprocedural images 150 and overload on the preprocedural images 150 to track movement of the real-time imaging system 130. Similarly, during the medical procedure, a tracked position of the intervention tool 180 may be mapped to coordinates in the preprocedural images 150 and overload on the preprocedural images 150 to track movement of the intervention tool 180.

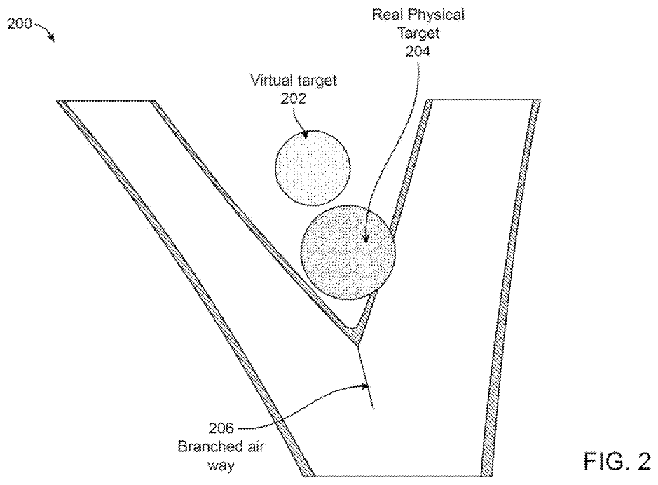

FIGS. 2-4 illustrate an example technique for calibrating registration of the real-time images 160 and preprocedural images 150. These examples are illustrated with respect to a procedure relating to a tumor proximate to a bronchial airway. However, the same principles may be applied with respect to other types of targets for a medical procedure in the context of airways or other anatomical structures. For example, similar techniques may be employed to calibrate registration with respect to other types of tumors or to target other tissue types that may be obscured (e.g., for targeting a breast cancer tumor obscured by breast tissue).

FIG. 2 shows a top view 200 of a branching peripheral airway. This view 200 shows a virtual target 202 at a virtual target position that corresponds to a position that was marked in the preprocedural images 150. The view 200 also shows the actual position of the real physical target 204 (e.g., a tumor). The physical target 204 is shown in FIG. 2 for illustrative purposes, but its exact position (i.e., the error relative to the position of the virtual target 202) is not initially known from the preprocedural images 150. Thus, an initial display of the preprocedural images 150 (e.g., rendered via the I/O device 170) may show only the branched airway 206 and the virtual target 202 without showing the real physical target 204. As can be seen, the position of the virtual target 202 obtained from the preprocedural image 150 is imprecise in that it does not exactly correspond to the position of the physical target 204.

Nevertheless, the virtual target 202 is generally close enough that it may be used for initial navigation of the real-time imaging system 130 to a vicinity of the physical target 204.

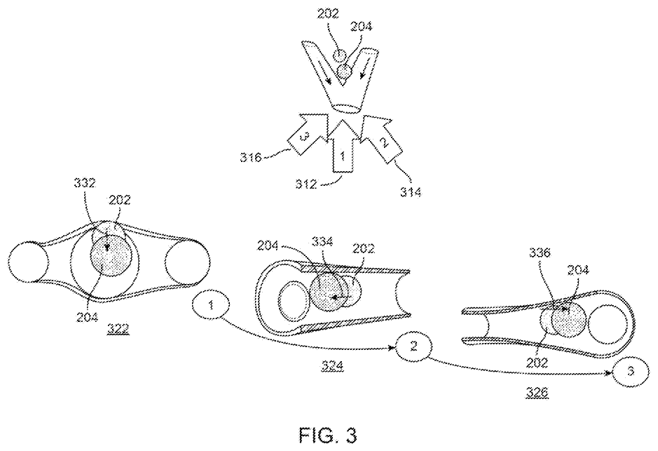

FIG. 3 illustrates an example embodiment of a process for calibrating the position of the virtual target 202. In this process, the medical targeting system 140 renders one or more views of the airway 206 and the physical target 204 from the real-time images 160 once the physical target 204 is within the field of view of real-time imaging system 130. These views may also render the virtual target 202 as a virtual object overlaid on the real-time images 160 at the virtual target position which has been registered to the image space of real-time images 160. In an embodiment, the position of the probe tip of the real-time imaging system 130 may similarly be mapped to coordinates in the preprocedural images 150 and may be displayed at its estimated position in a rendering of the preprocedural images 150.

The medical targeting system 140 may facilitate adjustment of the virtual target position of the virtual target 202 until it substantially overlaps with the real physical target 204. In an embodiment, the adjustments may be performed in multiple steps that each adjust the virtual target position of the virtual target 202 along a single axis. For example, in the illustrated example, the adjustment is performed in three steps. In step 1, the virtual target position of the virtual target 202 is observed from a first viewpoint 312 (as illustrated in view 322) and adjusted along a first axis 332 until the virtual target 202 overlaps with the observed physical target 204 in the real-time images 160 as seen from the first viewpoint 312. In step 2, the real-time imaging system 130 may then be adjusted to capture real-time images 160 from a second viewpoint 314 (as illustrated in view 324). The virtual target position of the virtual target 202 may then be further adjusted along as second axis 334 until it overlaps with the observed physical target 204 as seen from the second viewpoint 314. In step 3, the real-time imaging system 130 is once again adjusted to capture real-time images 160 from a third viewpoint 316 (as illustrated in view 326). The virtual target position of the virtual target 202 may then be further adjusted along a third axis 336 until it overlaps with the observed physical target 204 as seen from the third viewpoint 316. The first, second, and third axes 332, 334, 336 may be perpendicular or non-perpendicular axes. In an embodiment, the directions of the viewpoints 312, 314, 316 may correspond to the longitudinal axes of surrounding airway branches that physically constrain the range of viewpoints available to the real-time imaging system 130.

In an embodiment, the above-described adjustments to the virtual target position of the virtual target 202 may be performed manually in response to control inputs from a medical professional viewing the real-time images 160 with the overlaid virtual target 202 on a display (e.g., I/O device 170). The position of the virtual target 202 may be adjusted in substantially real-time such that the medical professional can observe the changes and continue to adjust position of the virtual target 202 until the medical professional deems that sufficient overlap is achieved from the desired number of viewpoints. In some embodiments, the virtual target 202 may be represented as a partially transparent object to enable the medical professional to concurrently view the virtual target and the actual physical target and certify the accuracy of the alignment.

In another embodiment, the medical targeting system 140 may apply image analysis at each step to detect when a sufficient overlap is achieved. The medical targeting system 140 may then alert the medical professional and recommend proceeding to the next step. In yet another embodiment, the adjustments may be performed automatically. Here, the position of the observed physical target 204 may be marked or automatically detected (via image analysis) in the real-time images 160. The medical targeting system 140 may then adjust the position of the virtual target 202 automatically until sufficient overlap is achieved. In the same manner described above, the adjustments may be performed over multiple steps from different viewpoints. The medical targeting system 140 may furthermore automatically determine when a sufficient number of views (and corresponding adjustments) are achieved for sufficient calibration. For example, the medical targeting system 140 may iterate adjustments until an absolute error between the two targets is below a defined threshold.

Once alignment is achieved, the calibrated virtual target position (which now more precisely corresponds to the position of the physical target 204), may be used to facilitate guidance of an intervention tool 180 to the target position to perform the medical procedure. As discussed above, the intervention procedure may be performed together with observed real-time images 160 or may be performed without access to real-time images 160 by relying only on the calibrated virtual target position. The position of the intervention tool 180 may be tracked and displayed as an overlay on the preprocedural images 150 by mapping its tracked coordinates to coordinates of the preprocedural images based at least in part on the registration.

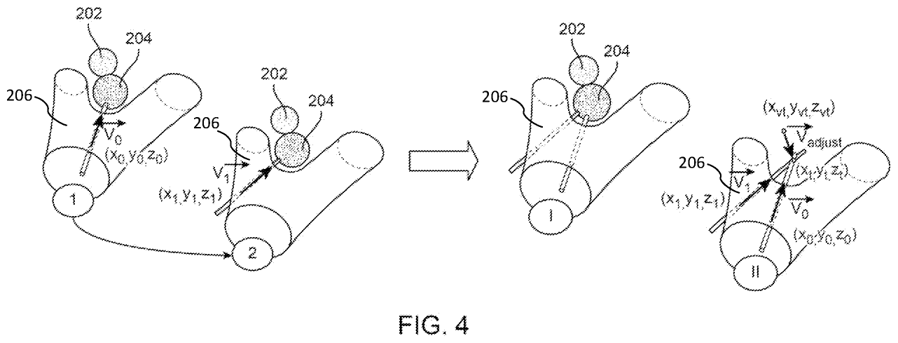

FIG. 4 illustrates another embodiment of a process for calibrating a virtual target position of a virtual target 202. FIG. 4 is shown using external views of an airway 206. As explained above, the position of the physical target 204 illustrated in FIG. 4 is not initially known from the preprocedural images 150 but is shown for illustrative purposes to demonstrate the calibration technique.

In this embodiment, a medical professional or the robotic guidance system 120 may control guidance of the real-time imaging system 130 to a predicted vicinity of the physical target 204 based at least in part on the virtual target position of the virtual target 202. Once the physical target 204 is identified in the real-time images 160, the real-time imaging system 130 may capture images of the physical target 204 from multiple viewpoints. The precise position and vector direction of the probe tip to the centroid of the physical target (defining the viewpoint of the images) may be recorded for each image. The position of the centroid of the physical target 204 may be manually marked by a medical professional viewing the real-time images 160 via the I/O device 170 or may be automatically identified using image processing techniques applied by the medical targeting system 140. Furthermore, a time-of-flight based system (e.g., using the NIRF sensor or lasers) may be used to estimate distance from the probe tip to the target 204. In the illustrated example, at least two such images are recorded: a first image from a first viewpoint with the probe tip located at a position (x₀, y₀, z₀) and oriented in a direction V₀, and a second image from a second viewpoint with the probe tip located at a position (x₁, y₁, z₁) and oriented in a direction V₁.

In an embodiment, the medical professional may manually choose these viewpoints (e.g., specific positions and/or orientations) for capturing the images. In another embodiment, the medical professional may control movement of the real-time image system 130 within the airway, and the medical targeting system 140 may automatically select the viewpoints for capturing the relevant images. For example, the medical targeting system 140 may determine the viewpoints based at least in part on predefined criteria for optimizing calibration. In another embodiment, the medical targeting system 140 may control the robotic guidance system 120 to automatically move the real-time imaging system 130 in a vicinity of the virtual target 202 until a sufficient number of viewpoints are captured that enable calculation of the centroid of the physical target within a degree of accuracy acceptable for the medical procedure being performed, or as otherwise specified by an operator. In some situations, two viewpoints may be deemed sufficient. In other embodiments, three or more viewpoints may be acquired before estimating the physical target position.

Once at least two viewpoints are captured, the medical targeting system 140 may automatically calculate the position of the centroid of the physical target 204 defined by the intersection of the directional vectors V₀, V₁ from the tip positions (x, y, z) to the centroid of the physical target 204. The medical targeting system 140 may then move the virtual target position of the virtual target 202 to correspond to the centroid of the observed physical target 204 in accordance with the distance vector V_{adjust} between the position of the virtual target 202 (registered from the preprocedural images) and the centroid of the observed target 204.

In various embodiments, the medical targeting system 140 may perform a similar process using more than two different viewpoints, which may increase accuracy of the calibration. For example, a third viewpoint may be utilized to check the accuracy of the calibration resulting from the first two viewpoints. Alternatively, the intersections of direction vectors V from more than two viewpoints may be used to determine the centroid position of the target 204. In further embodiments, one or more additional landmarks (beyond the physical target 204) may be identified and similarly used to calculate errors and improve calibration accuracy.

In a further embodiment, a representation of the physical target 204 may be overlaid on preprocedural images 150 (the direct images, a virtual model of the anatomy derived from the images, or both) showing the virtual target 202 with the relative positions determined from the registration process. The virtual target position can then be adjusted to match the position of the physical target 204 using any of the techniques described above.

Various data associated with the above-described registration and calibration processes may be stored and utilized to improve and/or speed up future registration and calibration. For example, for each procedure, the medical targeting system 140 may store information such as times between capturing the preprocedural images 150 and the real-time images 160, the types of preprocedural images 150 used, and various registration and calibration parameters. The stored data may then be used to provide initial condition data for future procedures that may speed up the calibration process.

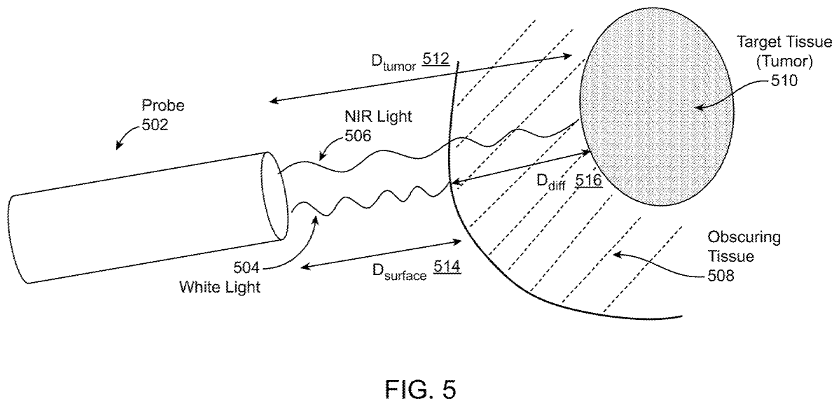

FIG. 5 illustrates an example technique for discerning between a target 510 (e.g., a tumor) and other obscuring tissue 508 (e.g., lung parenchyma) using the real-time imaging system 130. As described above, the probe tip 502 of the real-time imaging system 130 may include a NIRF fiberscope that emits near-infrared light 506 at one or more wavelengths and then detect the reflectance characteristics to discern whether or not the target 510 is present in the path of the near-infrared light 506. The wavelengths may be selected based at least in part on known reflectance characteristics of the target 510 and obscuring tissue 508 such that there is a discernable difference in reflectance properties for the different tissue types (i.e., such that the reflectance is much higher for the target tissue 510 than the obscuring tissue 508). For example, in a multispectral implementation, photons of two or more different wavelengths may be emitted and the resulting reflectance properties may be compared. In other embodiments, a hyperspectral technique may be employed in which the NIRF fiberscope sweeps the wavelength of emitted light over a range of wavelengths and the resulting reflectance waveform is analyzed to discern whether or not the beam intersects the target. In yet further embodiments, a reflectance value associated with a single wavelength may be sufficient to enable discernment between different tissue types.

Upon discerning the tumor or other tissue of interest 510, a time-of-flight computation may be performed to estimate the distance 512 from the probe tip 502 to the surface of the tumor 510. In one implementation, the time-of-flight computation may be applied in parallel with the discernment (i.e., the same reflected photons may be used to discern the tissue type and compute time-of-flight to estimate distance). In another implementation, the real-time imaging system 130 may first be configured to emit light of one or more wavelengths for the purpose of tissue discernment and may then be subsequently configured to perform a subsequent illumination to sense distance 512 based at least in part on time-of-flight.

As shown in FIG. 5, a white light camera that emits white light (which generally does not penetrate obscuring tissue 508) may furthermore be used to detect a distance 514 from the probe tip 502 to a surface of the obscuring tissue 508 based at least in part on a time-of-flight associated with the white light 504. A distance 516 between the surface of the obscuring tissue 508 and the surface of the tumor 510 may optionally be computed. This distance 516 may be utilized for intervention planning and/or during a procedure (e.g., when the probe tip 502 is no longer present). For example, the distance 516 may be used to determine how far a needle or other intervention tool should be extended through the obscuring tissue 508 to reach the tumor 510.

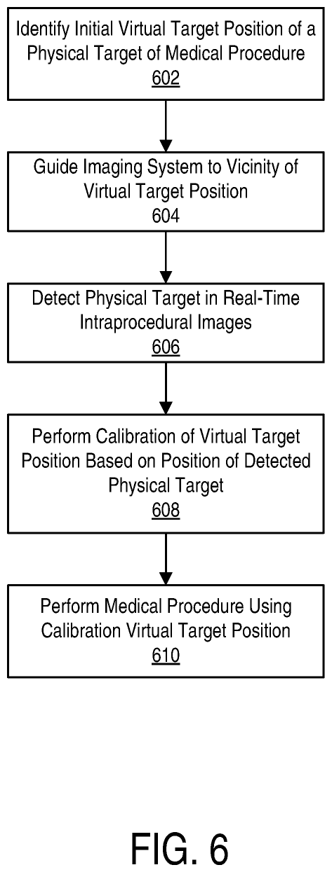

FIG. 6 is a flowchart illustrating an example embodiment of a process for performing a medical procedure. An initial virtual target position of a physical target of the procedure is identified 602 from preprocedural images of the patient. A real-time imaging system is guided 604 through one or more anatomical pathways to a vicinity of the initial virtual target position. Guidance may be based in part on registration between real-time images captured by the real-time imaging system and the preprocedural images. The physical target is detected 606 in the real-time intraprocedural images. As described above, the detection process may involve using a NIRF fiberscope and/or a white image camera to discern between the target tissue and obscuring tissue based at least in part on the detected reflectance properties associated with different tissue types. Responsive to detecting the physical target in the real-time images, a calibration is performed 608 to identify a calibrated virtual target position corresponding to a detected position of the physical target in the real-time images. Based at least in part on the calibrated virtual target position, a medical procedure is performed 610 in association with the physical target using an intervention tool 180.

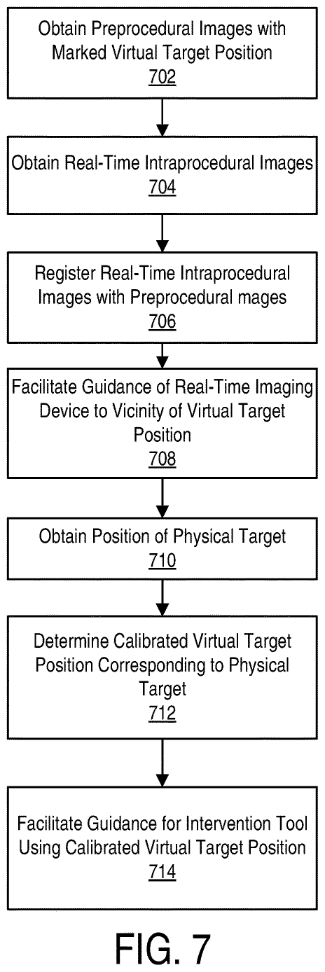

FIG. 7 is a flowchart illustrating an example embodiment of a calibration process associated with a medical procedure. A set of preprocedural images representing an anatomy are obtained 702 marked with a position of a virtual target. Once the patient is positioned for a medical procedure, real-time images of the anatomy are obtained 704 using a real-time imaging device. The real-time images are registered 706 to the preprocedural images to generate a mapping between coordinates of the real-time images and coordinates of the preprocedural images. The real-time imaging system 130 is electronically guided 708 to a vicinity of the virtual target position using the mapping. The position of the physical target is obtained 710 (e.g., by identifying the physical target in the real-time images). A calibrated virtual target position corresponding to the obtained position of the physical target is determined 712. Guidance is then facilitated 714 for an intervention tool to carry out the medical procedure using the calibrated virtual target position.

In further embodiments, the above-described systems and methods may be employed in contexts other than for medical procedures on real human patients. For example, the systems may be employed for simulated procedures on cadavers, corpses, or non-human animals for purposes such as product research and development, demonstrations, education, or medical training.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may include architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A method for performing guidance to a physical target, the method comprising:
obtaining preprocedural images representing an anatomy;
obtaining an initial virtual target position of the physical target in the preprocedural images;
obtaining, from a real-time imaging device, real-time intraprocedural images of the anatomy;
registering the real-time intraprocedural images of the anatomy to the preprocedural images to generate a mapping between coordinates of the real-time intraprocedural images and coordinates of the preprocedural images;
facilitating electronic guidance of the real-time imaging device to a vicinity of the initial virtual target position using the mapping;
obtaining a position of the physical target;
determining a calibrated virtual target position corresponding to the obtained position of the physical target; and
facilitating guidance of a medical tool to the physical target based at least in part on the calibrated virtual target position.

2. The method of claim 1, wherein determining the calibrated virtual target position comprises:
presenting, to a display device, a representation of a virtual target at the initial virtual target position overlaid on the real-time intraprocedural images;
receiving at least one user input for repositioning the virtual target and updating the display device to indicate the repositioning; and
determining the position of the physical target responsive to detecting alignment between a repositioned virtual target and the physical target.

3. (canceled)

4. The method of claim 1, wherein determining the calibrated virtual target position comprises:
generating a representation of a virtual target at the initial virtual target position overlaid on the real-time intraprocedural images;
automatically generating adjustments to the initial virtual target position and applying an image analysis until the virtual target substantially overlaps the physical target; and
determining the position of the physical target based at least in part on the adjustments.

5. The method of claim 1, wherein determining the calibrated virtual target position comprises:
receiving user inputs to control position of the real-time imaging device to capture at least two images of the physical target from different viewpoints; and
determining the position of the physical target based at least in part on the at least two images.

6-7. (canceled)

8. The method of claim 1, wherein facilitating the guidance comprises:
generating electronic navigation guidance for an electronically assisted intervention tool to the calibrated virtual target position.

9. The method of claim 1, wherein facilitating the guidance comprises:
displaying, on a display screen, the preprocedural images and a representation of a virtual target at the calibrated virtual target position overlaid on the preprocedural images; and
displaying, on the display screen, a position of an intervention tool overlaid on the preprocedural images to enable guidance of the intervention tool to the calibrated virtual target position.

10. The method of claim 1, wherein facilitating the guidance comprises:
displaying, on a display screen, the real-time intraprocedural images and a representation of a virtual target at the calibrated virtual target position overlaid on the real-time intraprocedural images.

11. The method of claim 1, wherein facilitating the guidance comprises:
determining a distance from an intervention tool to the physical target; and
generating control signals to guide the intervention tool based at least in part on the distance.

12. (canceled)

13. The method of claim 1, wherein the real-time imaging device comprises an endoscope for obtaining visible light images and wherein the real-time imaging device further includes a fiberscope for obtaining near-infrared fluorescent images, wherein registering the real-time intraprocedural images to the preprocedural images comprises:
registering the visible light images from the endoscope to the preprocedural images; and
registering the near-infrared fluorescent images from the fiberscope to the visible light images from the endoscope.

14. (canceled)

15. The method of claim 13, wherein the fiberscope has a substantially fixed relative position to the endoscope, and wherein registering the near-infrared fluorescent images to the visible light images is based at least in part on the substantially fixed relative position.

16. The method of claim 13, wherein the fiberscope is positioned within a field of view of the endoscope, and wherein registering the near-infrared fluorescent images to the visible light images is based at least in part on detecting the fiberscope in the visible light images or the near-infrared fluorescent images.

17. (canceled)

18. The method of claim 13, wherein the fiberscope is configured to pass through a working channel of the endoscope.

19. The method of claim 1, wherein obtaining the real-time intraprocedural images of the anatomy further comprises:
obtaining a time-of-flight of near-infrared fluorescent light projected by a fiberscope to determine a depth component of near-infrared fluorescent images.

20. The method of claim 1, wherein determining the calibrated virtual target position corresponding to the detected position of the physical target comprises:
emitting, by a fiberscope of the real-time imaging device, near-infrared light of one or more wavelengths;
detecting, by the fiberscope, reflectance properties of tissue in a path of the near-infrared light; and
discerning when the physical target is in the path of the near-infrared light based at least in part on the reflectance properties.

21. The method of claim 20, further comprising:
determining the detected position of the physical target based at least in part on a time-of-flight of the near-infrared light between the fiberscope and a surface of the physical target.

22. The method of claim 21, further comprising:
determining a distance from the real-time imaging device and a surface of obscuring tissue based at least in part on a time-of-flight of white light emitted from a white light illuminator of the real-time imaging device and reflected by the surface of the obscuring tissue; and
determining a distance from the surface of obscuring tissue and the surface of physical target.

23-28. (canceled)

29. A non-transitory computer-readable storage medium storing instructions for performing guidance to a physical target, the instructions when executed by one or more processors causing the one or more processors to perform steps including:
obtaining preprocedural images representing an anatomy;
obtaining an initial virtual target position of a physical target in the preprocedural images;
obtaining, from a real-time imaging device, real-time intraprocedural images of the anatomy;
registering the real-time intraprocedural images of the anatomy to the preprocedural images to generate a mapping between coordinates of the real-time intraprocedural images and coordinates of the preprocedural images;
facilitating electronic guidance of the real-time imaging device to a vicinity of the initial virtual target position using the mapping;
obtaining a position of the physical target;
determining a calibrated virtual target position corresponding to the obtained position of the physical target; and
facilitating guidance of a medical tool to the physical target based at least in part on the calibrated virtual target position.

30. (canceled)

31. A medical targeting system comprising:
a real-time imaging device;
a medical tool;
one or more processors; and
a non-transitory computer-readable storage medium storing instructions for performing guidance to a physical target, the instructions when executed by the one or more processors causing the one or more processors to perform steps including:
obtaining preprocedural images representing an anatomy;
obtaining an initial virtual target position of a physical target in the preprocedural images;
obtaining, from the real-time imaging device, real-time intraprocedural images of the anatomy;
registering the real-time intraprocedural images of the anatomy to the preprocedural images to generate a mapping between coordinates of the real-time intraprocedural images and coordinates of the preprocedural images;
facilitating electronic guidance of the real-time imaging device to a vicinity of the initial virtual target position using the mapping;
obtaining a position of the physical target;
determining a calibrated virtual target position corresponding to the obtained position of the physical target; and
facilitating guidance of the medical tool to the physical target based at least in part on the calibrated virtual target position.

32. The medical targeting system of claim 31, wherein the medical tool comprises an electronically navigated intervention tool, and wherein the medical targeting system further comprises:
a robotic control system for controlling electronic navigation of the electronically navigated intervention tool.

33. The medical targeting system of claim 31, wherein the real-time imaging device comprises an endoscope for obtaining visible light images.

34-42. (canceled)
